(19) United States
(12) Patent Application Publication
(10) Pub. No.: US 2009/0170933 A1
(43) Pub. Date: Jul. 2, 2009

(54) METHOD FOR REMOVING A MEDICAL DEVICE FROM A SPASMODIC CONSTRICTION IN A BODILY PASSAGEWAY

(75) Inventor: Michael E. Leckrone, Lafayette, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 12/341,440

(22) Filed: Dec. 22, 2008

Related U.S. Application Data
(60) Provisional application No. 61/016,686, filed on Dec. 26, 2007.

(51) Int. Cl. A61K 31/295; A61M 25/00; A61P 21/02

(52) U.S. Cl. 514/502; 604/514

(57) ABSTRACT

A method and apparatus for removing a medical device entrapped in a bodily passageway resulting from a spasmodic constriction of bodily tissue surrounding the device involves delivering to the site of the constriction an amount of an antispasmodic agent sufficient to relax the surrounding bodily tissue. The method may also be used for relaxing the tissue of a vessel wall to inhibit constriction of the vessel wall upon the introduction of a medical device into the vessel.

### RELATED APPLICATION

The present patent document claims the benefit of the filing date under 35 U.S.C. §119(e) of Provisional U.S. Patent Application Ser. No. 61/016,686, filed Dec. 26, 2007, which is hereby incorporated by reference.

### BACKGROUND OF THE INVENTION

1. Technical Field

The present invention relates to a method for treating bodily spasms around an inserted medical device. More particularly, the invention relates to a method for removing a medical device, such as a catheter or a sheath, from the site of a spasmodic constriction in a bodily passageway.

2. Background Information

Historically, in order to perform an invasive medical procedure, the physician was often required to cut one or more relatively large openings into the body of the patient in order to provide access to the target site. Typically, such openings were formed by cutting large areas of the body with a scalpel, and peeling back the skin and outer tissue in order to provide access to an inner organ or tissue.

In recent years, percutaneous procedures have replaced many of the classical open surgical techniques. In a percutaneous surgical procedure, the skin of the patient is initially punctured with a needle, and the target site within the body, such as a blood vessel, is accessed with the tip of the needle. A wire guide is then inserted through a bore of the needle into the vessel. Following removal of the needle, a medical device, such as a catheter or a sheath, is then inserted over the wire guide, and advanced through the vessel to the target area. The catheter or sheath may then be used to carry out the medical procedure, e.g., as a conduit for transmitting a fluid or a medical interventional device, such as a stent, into the vessel.

The use of percutaneous procedures in place of classical open surgical techniques has provided vast improvements the practice of medicine. Of primary importance is the fact that these techniques are much less intrusive to the patient. Not only is recovery time greatly reduced, but the overall pain and trauma endured by the patient during the medical procedures is considerably reduced in most instances. These benefits also typically reduce the overall costs of such procedures. In addition, the percutaneous procedures enable the physician to readily access areas of the body that previously could otherwise only be accessed by time consuming, intrusive, and often dangerous procedures.

Although percutaneous procedures have introduced significant advantages and efficiencies not previously available in the medical field, such techniques are not without at least some disadvantages. For example, in some instances, the introduction of a medical device, such as a catheter, sheath, or wire guide, into a blood vessel may cause the vessel to spasm. Such vasospasms may result in a constriction of the vessel around the device, referred to in the medical arts as a vasoconstriction. When the vessel constricts around the device, it becomes difficult, if not impossible, to move the device along the vessel. Such constriction around the device may prevent the procedure from being carried out, or if the procedure has already been carried out, may hinder or prevent removal of the device from the vessel. In severe instances, the vasoconstriction around the device may inhibit or prevent fluid flow through the vessel. This can result in ischemia, infarcation of tissue distal to the vasoconstriction, or necrosis. Such effects are clearly undesirable at any body vessel, but are particularly acute when they occur in small vessels, such as the cerebral vessels of the brain. In this instance, vasoconstriction around an inserted device can lead to a stroke or even death in a relatively short period of time.

It is desired to provide a method of treating a vascular spasm around a medical device that enables the physician to remove the device from the site of the spasm. It is also desired to provide a method of preventing the occurrence of a vascular spasm responsive to the introduction of a medical device into the vasculature of a patient.

### BRIEF DESCRIPTION OF THE DRAWINGS

FIG. 1 is a side view of one embodiment of an assembly suitable for use in the inventive method, comprising a perforated sheath positioned over a conventional guide catheter and dilator; and

FIG. 2 is an enlarged transverse cross-sectional view of the assembly taken along line 2-2 of FIG. 1

### DESCRIPTION OF PREFERRED EMBODIMENTS

For purposes of promoting an understanding of the present invention, reference will now be made to the embodiments illustrated in the drawings, and specific language will be used to describe the same. It should nevertheless be understood that no limitation of the scope of the invention is thereby intended, such alterations and further modifications in the illustrated assembly, and such further applications of the principles of the invention as illustrated therein being contemplated as would normally occur to one skilled in the art to which the invention relates.

In the following discussion, the terms “proximal” and “distal” will be used to describe the opposing axial ends of the assembly illustrated in the figures, as well as the axial ends of various component features of the assembly. The term “proximal” is used in its conventional sense to refer to the end of the assembly (or component thereof) that is closest to the operator during use of the assembly. The term “distal” is used in its conventional sense to refer to the end of the assembly (or component thereof) that is initially inserted into the patient, or that is closest to the patient during use.

When a medical device, such as a catheter, sheath, or wire guide, is introduced into a body duct, the presence of the foreign object (i.e., the medical device) in the body duct may cause the tissue surrounding the device to spasm. Such spasms may result in a constriction of the tissue of the duct around the device, thereby rendering it difficult, if not impossible, to move the device along the duct. Constrictions of this type around the device may prevent the intended medical procedure from being carried out, or if the procedure has already been carried out, may hinder or prevent removal of the device from the duct. When the duct is a body vessel, such as a blood vessel, constriction of the vessel around the device (vasoconstriction) may inhibit or prevent fluid flow through the vessel. As stated above, vasoconstriction may result in ischemia, infarcation of tissue distal to the vasoconstriction, or necrosis.

In one form, the present invention involves the introduction of an antispasmodic compound to the duct before a spasm has occurred. This minimizes the likelihood of a spasm by the tissue of the duct around the medical device. Alternatively, if a spasmodic episode has occurred, introduction of the antispasmodic compound facilitates further movement, such as removal, of the device from the duct. For simplicity but not by way of limitation, the duct will be referred to herein as a blood vessel. All blood vessels are included, but it is believed that the invention will be more applicable for use in arteries. Those skilled in the art will appreciate that the present invention is also applicable to other body vessels, as well as any other body ducts, such as the urinary ducts (urethra and ureters), into which a medical device may be inserted.

Introducer sheaths, guide catheters, wire guides, and like devices are well known in the medical arts for providing access to a blood vessel. Among the typical uses of such devices is as a conduit for transmitting a fluid or a medical interventional device, such as a stent, to a target site in the vessel. The medical arts have now advanced to the extent that such catheters, sheaths and wire guides generally have sufficient trackability and kink resistance to enable them to traverse tortuous passageways, and thereby reach target sites that had previously been difficult, if not impossible, to reach. Non-limiting examples of such catheters and sheaths are described in U.S. Pat. Nos. 5,380,304, 5,769,830, and 6,939,337, and U.S. Patent Publication No. 2001/0034514, among many other patent documents. The patent documents listed above are incorporated by reference as if fully rewritten herein.

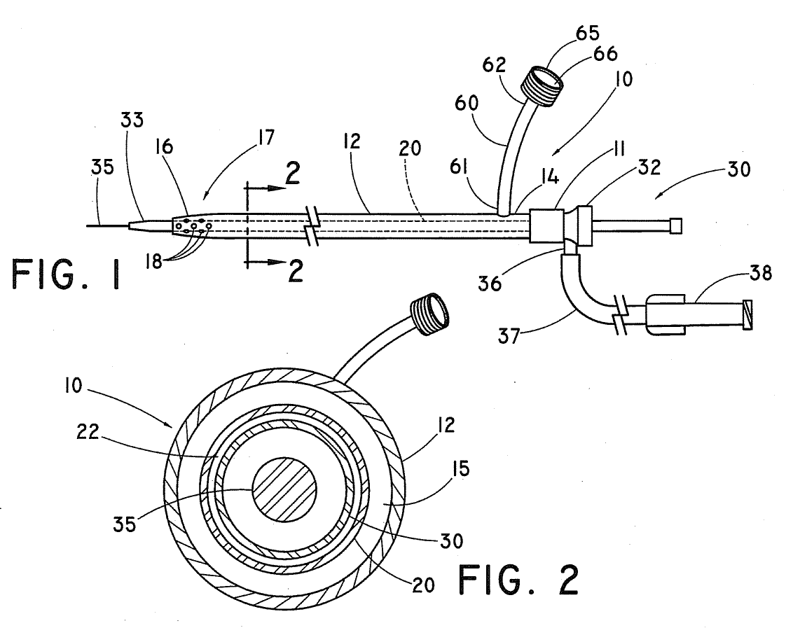

One non-limiting example of a suitable assembly 10 that is particularly appropriate for use in the inventive method is illustrated in FIGS. 1 and 2. In this embodiment, assembly 10 is formed of concentric tubular members, namely outer sheath 12 and inner catheter 20. Outer sheath 12 includes a proximal portion 14, a distal portion 16, and has a passageway, or lumen, 15 extending longitudinally therethrough. Preferably, distal portion 16 tapers to a tapered distal end 17. Outer sheath 12 can have any composition conventionally used for such purposes in the medical arts, such as nylon, polyethylene, polyurethane, etc. Non-limiting additional examples of suitable sheaths are further described in the incorporated-by-reference patents. A plurality of openings, such as sideports 18, may be disposed along a length of the sheath. In FIG. 1, sideports 18 are shown disposed along the distal portion of sheath 12; however, sideports may be positioned at any portion of the length of the sheath. Any number of sideports may be provided, and the sideports may have any geometrical configuration sufficient for the purposes described herein, such as round or elliptical.

A hub 11 may be provided at the proximal end 14 of sheath 12. Typically, hub 11 will include at least one valve, such as a conventional hemostatic valve, or one or more valve disks. A post-valve injection line 60 extends from outer sheath 12 to a conventional connector 65, such as a Luer lock connector. First end 61 of injection line 60 communicates with sheath inner passageway 15. Second end 62 of the injection line is connected to connector 65, for communication with a medical device (e.g., a syringe) for transmission of an antispasmodic medication as described. Connector 65 may include a septum 66 through which a needle tip (not shown) may be inserted to carry out an injection in well-known fashion.

Catheter 20 may be any conduit known in the art for transmitting fluids and/or medical devices to a target site. Non-limiting examples of suitable catheters are described in the incorporated-by-reference patent documents. Catheter 20 is positioned within central passageway 15 of outer sheath 12. Catheters are now routinely used in the medical arts for gaining access to a target site in the body, and those skilled in the art can readily select an appropriate catheter for a particular use. Such catheters may be multi-layered, and may include a reinforcing member, such as a flat wire or a braid. In addition, the catheter may have successive lengths having a durometer that differs from the durometer of an adjacent length, all in a manner well known in the art. Typically, the catheter will have segments of increasingly lower durometer (i.e., having greater flexibility) toward the distal end of the catheter.

The embodiment shown also includes an optional dilator 30 and connector hub 32. Dilators and connector hubs for use in combination with introducer devices, such as catheter 20, are well known in the art. The use of a dilator and hub is not required, and if such components are utilized, they may be replaced with various other dilators and hubs known in the art. As shown herein, dilator 30 extends longitudinally through the passageway 22 of catheter 20. The dilator includes a tapered distal end 33 for accessing and dilating a vascular access site, e.g., over a conventional wire guide 35 by any conventional vascular access technique, such as the well-known Seldinger technique. Connector hub 32 is attached about the proximal end of catheter 20 during use, and may also include one or more conventional valves, such as silicone disks (not shown), for preventing the backflow of fluids therethrough. Connector hub 32 may also include a side arm 36, to which a polymeric tube 37 and a Luer lock connector 38 may be connected for introducing and aspirating fluids therethrough in conventional fashion.

FIG. 2 is an enlarged transverse cross-sectional view of assembly 10. Those skilled in the art will appreciate that the relative placement of the tubular members and the wire guide shown in FIG. 2 are exemplary only, and are not meant to imply a required amount of spacing between the members and the wire guide. Rather, the relative dimensions of the components illustrated herein are selected such that, e.g., sufficient clearance is present between the tubular members to provide adequate space for transmittal of a desired volume of a drug, such as an antispasmodic agent.

The following example describes one possible use of the inventive method, in this case, for removing a medical device entrapped in a blood vessel resulting from a spasmodic constriction of bodily tissue surrounding the device. Initially, access is made to the interior of the blood vessel, e.g., by utilizing the Seldinger percutaneous access technique described above. In the Seldinger technique, the vessel is punctured with the tip of a small-bore needle. A wire guide is passed into the vessel through the bore of the needle, and the needle is thereafter removed. The distal end of a guide catheter assembly, such as assembly 10 illustrated in the figures herein, is inserted into the vessel over the wire guide, and is advanced into the vicinity of the constriction.

An antispasmodic agent is inserted into tubular line 60, such as by injection from a syringe (not shown) through septum 66 and connector 65, upstream of a constriction. The antispasmodic agent enters passageway 15, and traverses the length of outer sheath 12 within passageway 15 until it reaches sideports 18. The antispasmodic agent then passes through one or more of sideports 18 and into the bloodstream of the vessel. The flow of blood in the vessel carries the antispasmodic agent to the site of the constriction wherein, upon contact with the vessel wall, it acts in rapid fashion upon the vessel wall tissue to relax the constriction such that the medical device can be moved through the vessel.

The type, and amount, of the antispasmodic agent may be varied depending upon the particular bodily passageway in which the medical device has become entrapped. When the bodily passageway is a blood vessel, a vasodilator may be utilized. Preferably, the vasodilator should be a fast-acting compound, such as a nitric oxide donor compound. Nitric oxide donor compounds have a short half-life that is effective for rapidly dilating a vessel. Nitric oxide donor compounds are widely used as vasodilators, and a skilled artisan can readily select an appropriate one of these compounds for use herein. One particularly preferred nitric oxide donor compound is sodium nitroprusside. The amount of vasodilator used should, of course, be sufficient to achieve the intended effects.

In the non-limiting example provided above, the vasodilator was introduced into the blood stream to relax the tissue of the vessel wall that had already constricted about the medical device. The inventive method may also be favorably utilized in a preventative manner to minimize, or even prevent, the constriction of the vessel wall about the medical device in the first place. In this event, the vasodilator may be added to the vessel prior to, or contemporaneous with, the introduction of the medical device. If desired, the physician may include one or more other drugs with the antispasmodic agent, and inject the drugs into the vessel as a mixture, or “cocktail”. One non-limiting example of a type of drug that may be mixed with the antispasmodic agent and injected as part of a drug cocktail is a numbing agent. Those skilled in the art can readily select an appropriate numbing agent for a particular use. Members of the “caine” family of drugs, such as lidocaine and novacaine, are believed to be particularly appropriate for such use.

Utilizing the inventive method does not necessarily require the use of an assembly as shown in FIGS. 1 and 2. For example, a separate sheath 12 is not necessarily required. In this instance, the sideports would be provided along the designated length of the catheter 20, and the assembly would be constructed such that the vasodilator is passed through the passageway 22 of catheter 20. Passageway 22 is in communication with the vessel wall via the sideports.

While these features have been disclosed in connection with the preferred embodiments described above, other embodiments of the invention will be apparent to those skilled in the art that come within the spirit of the invention as defined in the following claims.

What is claimed is:

1. A method for removing a medical device entrapped in a bodily passageway resulting from a spasmodic constriction of bodily tissue surrounding said device, comprising:
delivering to a site of said spasmodic constriction an amount of an antispasmodic agent sufficient to relax said surrounding bodily tissue to permit removal of said medical device.

2. The method of claim 1, wherein the bodily tissue comprises a vessel wall, and the antispasmodic agent comprises a vasodilator.

3. The method of claim 2, wherein said vasodilator comprises a nitric acid donor compound.

4. The method of claim 2, wherein the vasodilator comprises sodium nitroprusside.

5. The method of claim 1, further including the steps of providing a guide catheter assembly having a size and shape suitable for introduction into said bodily passageway, said guide catheter assembly having at least one sideport along a distal portion thereof; inserting at least said distal portion of said guide catheter assembly into said bodily passageway; and delivering said antispasmodic agent into said passageway through said at least one sideport.

6. The method of claim 5, wherein said guide catheter assembly comprises inner and outer concentric tubular members, said guide catheter assembly structured such that said at least one sideport is disposed in said outer tubular member, and said antispasmodic agent is introduced in a space between said tubular members for passage through said sideport to said site.

7. A method for relaxing tissue of a vessel wall to inhibit a constriction of said vessel wall upon introduction of a medical device in said vessel, comprising:
delivering into said vessel a sufficient amount of an antispasmodic agent at least contemporaneous with the introduction of said medical device to relax the tissue of said vessel wall and inhibit said vessel wall constriction.

8. The method of claim 7, wherein said antispasmodic agent comprises a nitric acid donor compound.

9. The method of claim 7, further comprising the step of mixing said antispasmodic agent with another agent to form a mixture, and delivering said mixture into said vessel.

10. The method of claim 9, wherein said antispasmodic agent comprises a nitric acid donor compound, and said other agent comprises a numbing agent.
